(19) United States
(12) Patent Application Publication
(10) Pub. No.: US 2010/0319685 A1
(43) Pub. Date: Dec. 23, 2010

(54) MEDICAL LIQUID DROPLET APPARATUS

(75) Inventor: NENG-CHIH YU, Taipei County (TW)

Correspondence Address: WPAT, PC;INTELLECTUAL PROPERTY ATTORNEYS, 7225 BEVERLY ST., ANNANDALE, VA, 22003, US

(73) Assignee: QUATEK CO., LTD., Taipei City (TW)

(21) Appl. No.: 12/871,299

(22) Filed: Aug. 30, 2010

Related U.S. Application Data
(63) Continuation-in-part of application No. 12/410,647, filed on Mar. 25, 2009.

(30) Foreign Application Priority Data
Mar. 25, 2008 (TW) 097110475

(51) Int. Cl. A61M 11/00

(52) U.S. Cl. 128/200.16; 128/200.14

(57) ABSTRACT

The present invention provides a medical liquid droplet apparatus, which comprises a driving base and a polymeric film. The driving base is capable of providing a vibrating energy. The polymeric film, having a plurality of tiny openings formed thereon, is disposed on the driving base for receiving the vibrating energy so as to generating liquid droplet. The present invention further comprises a first seal element and a second seal element respectively and hermetically coupled to a first side and a second side of the medical liquid droplet apparatus while the medical liquid droplet apparatus is packaged within a chassis. By means of the seal elements provided in the present invention, it is capable of keeping the nebulization efficiency and improving the convenience during the packaging process so as to reduce the manufacturing cost.

### FIELD OF THE INVENTION

The present invention relates to a nebulization technique, and more particularly, to a medical liquid droplet apparatus having a polymeric film as a vibrating film for breaking up liquid into droplets, and assembled hermetical seal for facilitating assembly.

### BACKGROUND OF THE INVENTION

A patient, who suffers from asthma or a chronic bronchial disease, may have to take an inhalation therapy, for example, to take a bronchiectatic spray when getting a trouble in breathing. Various nebulizers have been disclosed for nebulizing liquid medicine, enabling nebulized medicine to be easily inhaled by the patient, whereas the nebulization speed can be an important index for measuring the performance of an individual nebulizer.

Conventional nebulizers can be divided into two categories: high-efficiency and reduced-efficiency. A high-efficiency nebulizer usually is a bulky device that can nebulize a solution with a comparably better nebulization speed, but at the cost of more electricity consumed. On the other hand, a reduced-efficiency nebulizer usually is a small device that can nebulize a solution using less electricity, but at an inferior nebulization speed instead. Although the aforesaid two types of nebulizers have their respective pros and cons, generally they all adopt a nebulization element that is made of metal, as the liquid atomizing apparatus disclosed in U.S. Pat. No. 6,863,224 that is shown in FIG. 1A. In FIG. 1A, the vibration of an ultrasonic vibration source 10 is transmitted to a nebulization element 11 for causing the same to vibrate correspondingly. In addition, as the nebulization element 11 is configured with a plurality of openings, the nebulization element 11 is capable of nebulizing liquid into mist as it is vibrating. It is noted that the nebulization element 11 used in the aforesaid liquid atomizing apparatus is manufactured by an electroforming means using a NiPD alloy.

In addition, U.S. Pat. No. 6,651,650 disclosed a nebulizer having a polymeric film for breaking up the liquid into droplets. Although it teaches a way for generating droplets by means of the polymeric film, the film is actuated to vibrate by reciprocated force punching directly on the film. According to the disclosure in this art, the pump shaft is vibrated in the axial direction by vibration of the ultrasonic vibrator, whereby the liquid inside the liquid vessel is pumped up through the pump bore of the pump shaft. Since the mesh plate is urged against the upper end face of the pump by the resilient member, the mesh plate also vibrates by following up the motion of the pump shaft. Owing to the fact that the mesh plate is vibrated, and by virtue of the fact that the mesh plate is biased by the resilient member, the mesh plate acts as a type of valve which opens and closes the opening at the upper end of the pump bore in the pump shaft. According to the foregoing description, the mesh plate vibrates due to the push of the shaft and the reciprocating motion of the resilient member such that the liquid pumped up from the shaft can be atomized and sprayed. In this art, there has a drawback that the polymeric film is urged by the upper surface of the pump shaft so that the polymeric film, unlike the metal film, in this art is easily broken; thereby malfunction of the polymeric film is occurred.

Please refer to FIG. 1B, which illustrates a nebulizer package of the prior art. The nebulizer 12 comprises a vibrating source 120, an energy conductor 121, being arranged at a side of the vibrating source 120, having a hole 122 and made of a metal material for conducting a vibrating energy generated by the vibrating source 120, and a metal film 123, disposed on the energy conductor 121, having a plurality of tiny openings 124 corresponding to the hole 122. The vibrating source 120 further comprises two electrical terminals 125 and 126 respectively disposed at the upper and bottom sides of the vibrating source 120. The two electrical terminals 125 and 126 are coupled to a power source so that the vibrating source 120 is capable of receiving the power to vibrate.

It is known that if the medical liquid is leaked, the electrical terminal 125 and 126 of the vibrating source 120 will be short-circuited, thereby affecting the operation of the nebulizer 12. In order to prevent medical liquid from leakage, a waterproof material 13 such as epoxy or waterproof resin is coated on the upper and bottom side of the vibrating source 120 and energy conductor 121 before packaging the nebulizer 12. Although the waterproof material 13 is capable of preventing the electrical terminals 125 and 126 from being short-circuited, or being contaminated by the medical liquid or droplets, the nebulizer 12 has to be abandoned due to the malfunction for generating droplets caused by improper coating process, such as the waterproof material obstructing the tiny openings, for example. Even if the improper coating can be removed by some process, it takes time and is inconvenient for the package process, and further increases the production cost as well. Besides, since the waterproof material covers the vibrating source and energy conductor, the vibrating energy will be absorbed by the waterproof material so as to decrease the vibrating energy received by the metal film, thereby reducing the nebulization efficiency of the nebulizer.

Therefore, it is in great need to have a medical liquid droplet apparatus that is not troubled by the aforesaid problems.

### SUMMARY OF THE INVENTION

The present invention provides a medical liquid droplet apparatus which is packaged by hermetical seals so that the medical liquid droplet apparatus not only can maintain the nebulization efficiency but also improve convenience during assembly process and reduce the production cost in the mean time.

The present invention is to provide a medical liquid droplet apparatus having a polymeric film configured therein to be used as its nebulizing film, which can prevent the medical liquid droplet apparatus from malfunctioning due to the medicine eroding so as to maintain the polymeric film to function normally for a specific period of time.

The present invention is to provide a medical liquid droplet apparatus having a polymeric film configured therein to be used as its nebulizing film, which can prevent the embrittled problem due to the high frequency vibration occurred in the conventional metallic nebulizing film from happening so as to maintain the polymeric film to function normally for a specific period of time.

The present invention is to provide a medical liquid droplet apparatus comprising a polymeric film which has a plurality of tiny openings formed thereon, wherein diameter of each tiny opening is between 2˜6 μm, and center-to-center distance between the adjacent two tiny openings is between 60˜180 μm. By means of the arrangement of the foregoing opening characteristics, it is capable of generating a better size of the droplet and sufficient spray quantity so as to be suitable for being utilized in the medical field.

In an exemplary embodiment of the invention, a medical liquid droplet apparatus is provided, which comprises: a vibrating source comprising a first surface and a second surface oppositely corresponding to the first surface for providing a vibrating energy, wherein the first surface and second surface respectively has an electrical terminal formed thereon; an energy conductor, comprising a third surface connected to the second surface, a fourth surface oppositely corresponding to the third surface, and a hole opened through the third surface and the fourth surface, for conducting the vibrating energy; a polymeric film, disposed on the energy conductor, having a plurality of tiny openings corresponding to the hole for receiving the vibrating energy, thereby breaking up the liquid into droplets; a first hermetical seal having a first protrusion part abutting against the third surface for separating the electrical terminal on the first surface from the droplets, and a first through hole corresponding to the plurality of tiny openings; and a second hermetical seal having a second protrusion part abutting against the fourth surface for separating the electrical terminal on the second surface from the liquid, and a second through hole corresponding to the hole.

### BRIEF DESCRIPTION OF THE DRAWINGS

The present invention will become more fully understood from the detailed description given herein below and the accompanying drawings which are given by way of illustration only, and thus are not limitative of the present invention and wherein:

FIG. 1A is a cut-away perspective view of a liquid atomizing apparatus disclosed in U.S. Pat. No. 6,863,224.

FIG. 1B is a cross-sectional view illustrating a nebulizer package of the prior art.

FIG. 2A is a top view of a medical liquid droplet apparatus according to a first embodiment of the invention.

FIG. 2B is a cross-sectional view of the medical liquid droplet apparatus according to the first embodiment of the invention.

FIG. 3A is a cross-sectional view of the second embodiment of the medical liquid droplet apparatus of the present invention.

FIG. 3B illustrates a perspective view of the vibrating source of the present invention.

FIG. 4 is a cross-sectional view illustrating a package assembly of the medical liquid droplet apparatus of the present invention.

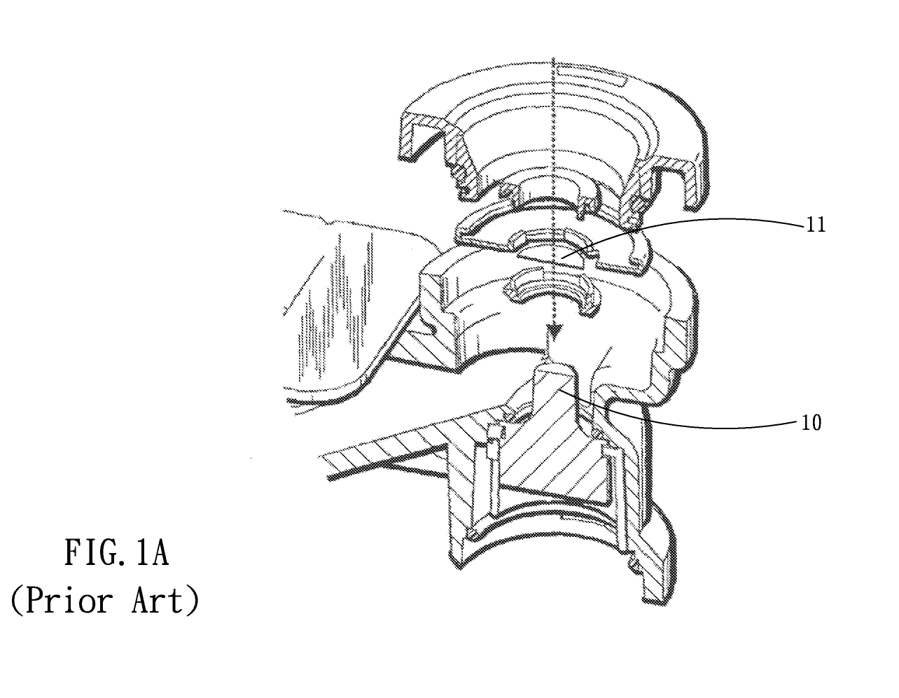

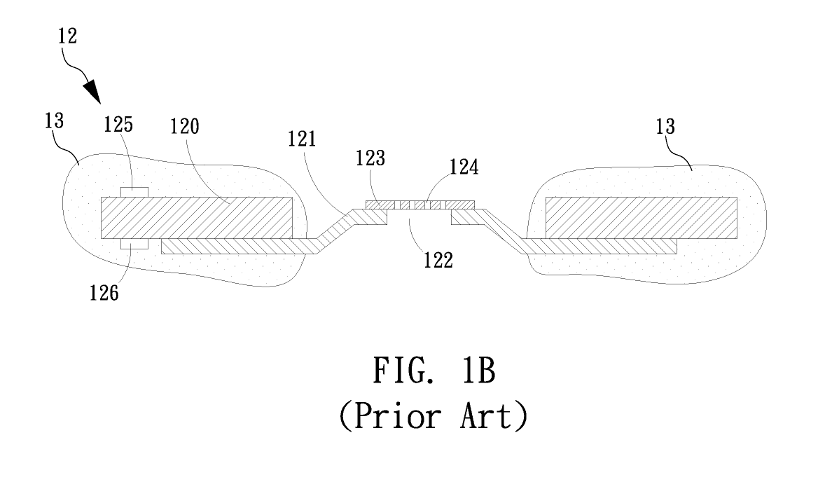

### DESCRIPTION OF THE EXEMPLARY EMBODIMENTS

For your esteemed members of reviewing committee to further understand and recognize the fulfilled functions and structural characteristics of the invention, several exemplary embodiments cooperating with detailed description are presented as the follows.

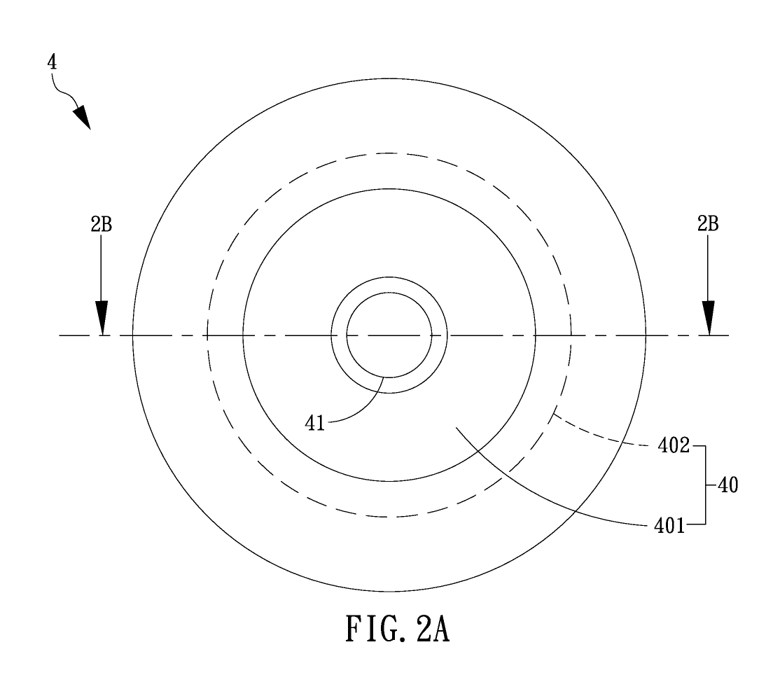

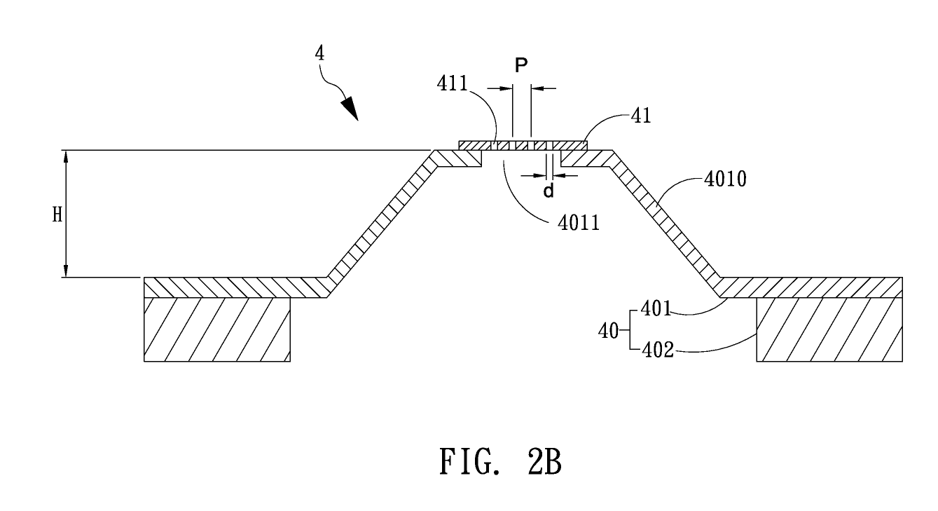

Please refer to FIG. 2A and FIG. 2B, which are a top view and a cross sectional view of a medical liquid droplet apparatus according to a second embodiment of the invention. In this first embodiment, the medical liquid droplet apparatus 4 comprises a driving base 40 and a polymeric film 41. The driving base 40, used for providing a vibrating energy, is further comprised of: a vibrating source 402 and an energy conductor 401, in which the energy conductor 401 is connected with the polymeric film 41 and is configured with a hole 4011 for receiving a liquid to be nebulized. In this second embodiment, the metallic energy conductor 401 is made of stainless steel, but is not limited thereby. Moreover, the vibrating source 402, being connected to the energy conductor 401, is capable of generating vibration for providing the vibrating energy. In this second embodiment, the vibrating source 402 can be a device selected from the group consisting of an ultrasonic vibration unit and a piezoelectric vibration unit; and is circularly disposed at the bottom of the energy conductor 401. It is noted that the means for driving the vibrating source 402 to vibrate is known to those skilled in the art and thus is not described further herein.

In FIG. 2B, the energy conductor 401 is further configured with a cone structure 4010 for raising the level of the energy conductor 401 connecting to the polymeric film 41 above the level of the same connecting to the vibrating source 402 by a specific height H. The polymeric film 41, which is configured with a plurality of tiny openings 411, is mounted on the driving base 40 for locating the plural tiny openings 411 at positions corresponding to the hole 4011 of the energy conductor 401. The polymeric film 41 is used for receiving the vibrating energy so as to generating liquid droplets. In this embodiment, the polymeric film 41 is made of a plastic, but is not limited thereby. As for the principle for causing the polymeric film 41 to vibrate, it is the same that described in the first embodiment and thus is not described further herein. In FIG. 2B, the diameter d of each tiny opening 411 is between 2 ˜6 μm while the center-to-center distance P between the adjacent two tiny openings 411 is between 60˜180 μm. According to the arrangement with respect to the foregoing opening diameter d and pitch distance P, the polymeric film is capable of producing appropriate particle size of droplet and sufficient spray quantity suitable for providing desired medication effects for the application in medical field such as inhalation therapy for lung, respiratory or pulmonary disease.

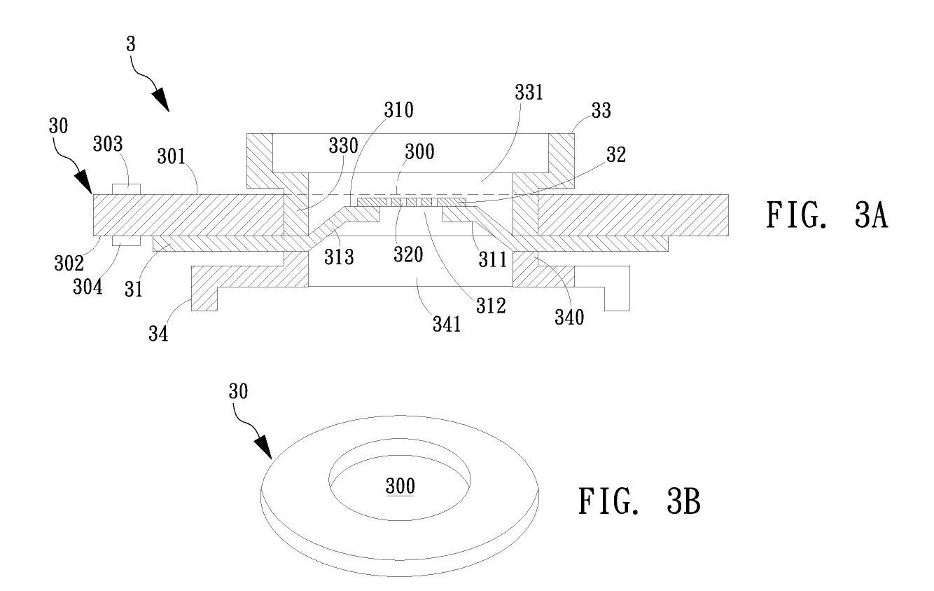

Please refer to FIG. 3A, which illustrates a second embodiment of the medical liquid droplet apparatus of the present invention. The medical liquid drop let apparatus 3 comprises a vibrating source 30, an energy conductor 31, a polymeric film 32, a first hermetical seal 33, and a second hermetical seal 34. Please refer to FIG. 3B, the vibrating source 30, in the present embodiment, is a ring-like structure having a hollowed area 300 formed therein. It is known in the art that the vibrating source 30 can be, but should not limited to, a supersonic vibrating element or piezoelectric vibrating element, wherein the vibrating source 30 is a piezoelectric vibrating element in the present embodiment. Referring back to FIG. 3A, the vibrating source 30 has a first surface 301 and a second surface 302 oppositely corresponding to the first surface 301. The first surface 301 and the second surface 302 respectively has an electrical terminal 303 and 304 formed thereon. The electrical terminals 303 and 304 are coupled to a voltage source whereby the vibrating source 30 is capable of receiving the power provided by the voltage source, thereby vibrating to provide a vibrating energy.

The energy conductor 31, coupled to the vibrating source 30 for receiving and conducting the vibrating energy, has a third surface 310, a fourth surface 311 oppositely corresponding to the third surface 310, and a hole 312 opened through the third surface 310 and the fourth surface 311. In the present embodiment, the third surface 310 is connected to the second surface 302 of the vibrating source 30. In the present embodiment, the energy conductor 31 is a ring-like structure. In addition, the energy conductor 31 further has a cone structure 313 formed around the hole 312 and accommodated within the hollowed area 300. The energy conductor 31 is made of a metal material, which is a stainless steel in the present embodiment.

The polymeric film 32, disposed on the energy conductor 31, has a plurality of tiny openings 320 corresponding to the hole 312. The polymeric film 32 receives the vibrating energy conducted by the energy conductor 31, thereby breaking up a liquid into droplets. In the present embodiment, the diameter of each tiny opening 320 is between 2˜6 μm while the center-to-center distance between the adjacent two tiny openings 320 is between 60˜180 μm. According to the arrangement with respect to the foregoing opening diameter and pitch distance, the polymeric film 32 is capable of producing fine droplets having specification of MMAD (mass median aerodynamic diameter)<5 μm. The fine droplet size and sufficient spray quantity are suitable for providing desired medication effects for the application in medical field such as inhalation therapy for lung, respiratory or pulmonary disease. The polymeric film 32 can be, but should not limited to, a plastic, Teflon and polyethylene (PE). In the present invention the polymeric film 32 is a plastic film.

The first hermetical seal 33 comprises a first protrusion part 330, which has a first through hole 331 corresponding to the plurality of tiny openings 32. The first protrusion part 330 is arranged in the hollowed area 300 and abuts against the third surface 310 of the energy conductor 31 so as to separate the electrical terminal 303 on the first surface 301 from the droplets discharged from the polymeric film 32. In the present embodiment, the first hermetical seal 33 is, but should not be limited to, a flexible rubber. The second hermetical seal 34 has a second protrusion part 340 abutting against the fourth surface 311 of the energy conductor 31 so as to separate the electrical terminal 304 on the second surface 302 of the vibrating source 30 from the liquid. The second hermetical seal 34 further has a second through hole 341 corresponding to the hole 312. In the present embodiment, the second hermetical seal 34 is, but should not be limited to, a flexible rubber.

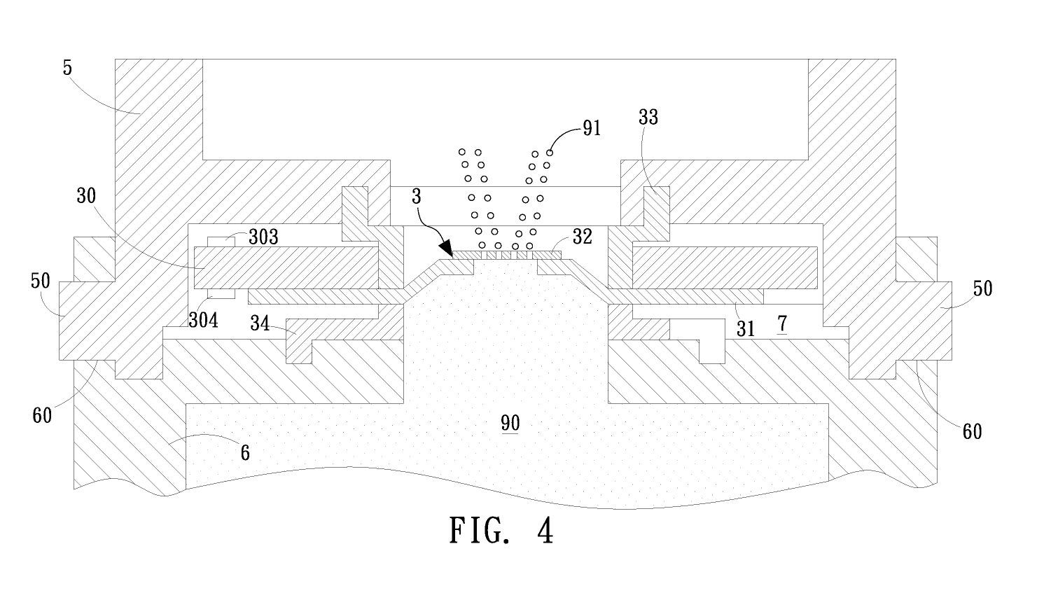

Please refer to FIG. 4, which illustrates an assembly package of the medical liquid droplet apparatus of the present invention. In the present embodiment, a first chassis 5 and a second chassis 6 is respectively arranged on an upper side and bottom side of the medical liquid droplet apparatus 3. The first chassis 5 covers and abuts against the first hermetical seal 33, while the second chassis 6 covers and abuts against the second hermetical seal 34. The first chassis 5 and the second chassis 6 are securely coupled to each other so as to define an accommodating space 7 such that the medical liquid droplet apparatus 3 can be hermetically sealed within the accommodating space 7. It is noted that the first chassis 5 has a plurality of latch knobs 50 for embedding into mating hole 60 of the second chassis 6 so that the first chassis 5 can be secured with the second chassis 6.

By means of the first hermetical seal 33 and the second hermetical seal 34, the electrical terminals 303 and 304 can be effectively separated from the liquid 90 and droplets 91. In addition, when the medical liquid droplet apparatus 3 is packaged within the first and second chassis 5 and 6, if hermetical effect or nebulization effect can't conform with the standard requirement during the process of quality control, it is capable of dismantling the first and second chassis 5 and 6 and adjusting the relative position between the medical liquid droplet apparatus 3, and the first hermetical seal 33 and the second hermetical seal 34. Therefore, the drawbacks of coating waterproof material, such as epoxy or waterproof resin, on the medical liquid droplet apparatus in the prior arts can be avoided. In the prior arts, the nebulizer has to be abandoned due to the malfunction for generating droplets caused by improper coating process such that the production cost is increased. Even if the improper coating can be removed and reworked, it will also increase inconvenience to the production line and raise the production cost. Besides, since the contact area between the first hermetical seal 33 and the second hermetical seal 34 and the energy conductor 31 is very small, less vibrating energy conducted by the energy conductor will be absorbed so that the nebulization efficiency will not be reduced comparing to the medical liquid droplet apparatus sealed by the waterproof material.

To sum up, the present invention provides a medical liquid droplet apparatus having a polymeric film configured therein to be used as its nebulizing film, which not only can prevent the medical liquid droplet apparatus from malfunctioning due to the medicine eroding, but also can prevent the embrittled problem due to the high frequency vibration occurred in the conventional metallic nebulizing film from happening, so that it can ensure its polymeric film to function normally for a specific period of time.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A medical liquid droplet apparatus, comprising:
a vibrating source comprising a first surface and a second surface oppositely corresponding to the first surface for providing a vibrating energy, wherein the first surface and second surface respectively has an electrical terminal;
an energy conductor, comprising a third surface connected to the second surface, a fourth surface oppositely corresponding to the third surface, and a hole opened through the third surface and the fourth surface, for conducting the vibrating energy;
a polymeric film, disposed on the energy conductor, having a plurality of tiny openings corresponding to the hole for receiving the vibrating energy, thereby breaking up the liquid into droplets;
a first hermetical seal having a first protrusion part abutting against the third surface for separating the electrical terminal on the first surface from the droplets, and a first through hole corresponding to the plurality of tiny openings; and
a second hermetical seal having a second protrusion part abutting against the fourth surface for separating the electrical terminal on the second surface from the liquid, and a second through hole corresponding to the hole.

2. The medical liquid droplet apparatus of claim 1, wherein the energy conductor is made of a metal.

3. The medical liquid droplet apparatus of claim 1, wherein the vibrating source is a device selected from the group consisting of an ultrasonic vibration element and a piezoelectric vibration element.

4. The medical liquid droplet apparatus of claim 1, wherein the vibrating source is substantially a ring-like structure having a hollowed area formed therein.

5. The medical liquid droplet apparatus of claim 1, wherein the energy conductor is substantially a ring-like structure.

6. The medical liquid droplet apparatus of claim 4, wherein the energy conductor further comprises a cone structure accommodated in the hollowed area.

7. The medical liquid droplet apparatus of claim 4, wherein the first protrusion part of the first hermetical seal is arranged in the hollowed area and abuts against the third surface.

8. The medical liquid droplet apparatus of claim 1, wherein polymeric film is made of a material selected from the group consisting of plastic, Teflon and polyethylene (PE).

9. The medical liquid droplet apparatus of claim 1, wherein diameter of each tiny opening is between 2 μm and 6μm.

10. The medical liquid droplet apparatus of claim 1, wherein center-to-center distance between the adjacent two tiny openings is between 60 μm and 180μm.

11. The medical liquid droplet apparatus of claim 1, further comprising a first chassis abutting against the first hermetical seal.

12. The medical liquid droplet apparatus of claim 11, further comprising a second chassis abutting against the first hermetical seal and coupled to the first chassis.
